(19) United States
(12) Patent Application Publication
(10) Pub. No.: US 2026/0115253 A1
(43) Pub. Date: Apr. 30, 2026

(54) VISCOUS COMPOSITION FOR TREATING ISCHEMIA

(71) Applicant: National Cheng Kung University, Tainan City (TW)

(72) Inventor: Lynn L.H. Huang, Tainan City (TW)

(21) Appl. No.: 18/988,352

(22) Filed: Dec. 19, 2024

Related U.S. Application Data
(63) Continuation of application No. 16/642,273, now abandoned.
(60) Provisional application No. 62/553,269, filed on Sep. 1, 2017.

(51) Int. Cl. A61K 38/18; A61K 31/352; A61K 31/4365; A61K 47/36; A61P 9/10

(52) U.S. Cl. CPC A61K 38/1866; A61K 31/352; A61K 31/4365; A61K 47/36; A61P 9/10

(57) ABSTRACT

A pharmaceutical composition for treating an ischemic tissue, comprising: a core component and a matrix component, wherein the core component includes a thrombolytic drug and the matrix component includes a hyaluronan or derivative thereof, the matrix component having a viscosity greater than 10 mPa·s.

### CROSS-REFERENCE TO RELATED APPLICATIONS

This application is a continuation of U.S. application Ser. No. 16/642,273, filed Feb. 26, 2020, which is the National Stage of International Application No. PCT/US2018/049003, filed on Aug. 31, 2018, which claims the benefit of U.S. Provisional Application No. 62/553,269, filed on Sep. 1, 2017. The contents of all prior applications are hereby incorporated by reference herein.

### BACKGROUND

In ischemia, the blood content of an organ or tissue is reduced. Ischemia can be a local manifestation of systemic anemia or a result of local blood circulation disorders. Types of ischemia include: 1) Compression ischemia can be caused by pressures on the arterial blood vessels from, for example, tumors, tight bandage and effusion, resulting in narrowing or occlusion of the lumen of the blood vessel. Clinically, hemorrhoids or ulcers formed from prolonged lying are instances of tissue necrosis caused by ischemia due to compression of lateral blood vessels, which can lead to muscle damages. 2) Obstructive ischemia from arterial thrombosis or embolism can lead to vascular occlusion, resulting in blocked blood supply to, for example, the limbs or heart. 3) Lateral limb ischemia can be caused by a rapid flow of a large amount of blood into the abdominal organs, resulting in ischemia of other organs and tissues.

Patients with peripheral arterial diseases of the lower limbs are mostly over 60 years old, and about one-half of the patients have diabetes. At present, angiogenesis treatments for ischemic lower limbs are being marketed. For example, the AutoloGel system is a wound dressing prepared by extracting a patient's autologous high-concentration plate-rich plasma (PRP) and adding a growth factor that promotes wound healing and a cytokine to form a gelatinous substance. However, such treatments are only used to treat chronic wounds, but they are not able to treat the underlying ischemia. Other treatments such as bypass grafting, vasodilation, and placement of vascular stents are necessary to resolve vascular occlusion.

Many studies on ischemic lower limbs are actively developing angiogenic therapeutics, such as cytokines or recombinant growth factors associated with angiogenic signaling, such as VEGF and FGF to stimulate angiogenesis. Platelet-derived growth factor (PDGF) has been found to stimulate mesenchymal cell proliferation, migration and differentiation in developmental or adult tissues, and is used to promote the release of endothelial-derived cells from bone marrow of patients to achieve vascular proliferation. Human umbilical vein endothelial cells (HUVEC) can also be stimulated by substances that are indirectly related to angiogenic signaling to stimulate angiogenesis. Treatment using tissue plasminogen activator (tPA) and HUVEC is given to increase the number of endothelial progenitor cells that migrate from the bone marrow to the blood vessels to promote vascular endothelial rejuvenation to achieve therapeutic effects.

The physiological condition of hyperglycemia caused by diabetes reduces the secretion of endothelial growth factor which, in the cases of severe vascular diseases, can lead to amputations. Most of the current treatment methods involve angiogenic factors, which face many difficulties in clinical use or medical efficacy. Nowadays, there is still no effective therapy to regenerate ischemia tissues, nonetheless to rescue limbs from amputation. Therefore, the development of a therapeutic composition for ischemia tissues suitable for most patients is an important problem to be solved.

### SUMMARY

In one aspect, described herein is a pharmaceutical composition for treating an ischemic tissue, comprising a core component and a matrix component, wherein the core component includes a thrombolytic drug and the matrix component includes a hyaluronan or derivative thereof, the pharmaceutical composition having a viscosity greater than 10 mPa·s. In some embodiments, the viscosity is 10 to 10000 mPa·s. In some embodiments, the pharmaceutical composition contains 1 mg/ml to 100 mg/ml of the hyaluronan.

In some embodiments, the hyaluronan has a mean molecular weight of 100 kDa to 5000 kDa. For example, the hyaluronan can have a mean molecular weight of 700 kDa to 2000 kDa.

In some embodiments, the viscosity of the pharmaceutical composition is within the range of viscosity of 3 to 10 mg/ml of hyaluronan that has a mean molecular weight of 700 to 2000 kDa. In some embodiments, the viscosity is the same as the viscosity of 5 mg/ml of hyaluronan having a mean molecular weight of 1560 kDa. The mean molecular weight of the hyaluronan can be 700 to 2000 kDa and the concentration of the hyaluronan can be 3 to 10 mg/ml. In some embodiments, the mean molecular weight of the hyaluronan is 1560 kDa and the concentration of the hyaluronan is 5 mg/ml.

In some embodiments, the matrix component in the pharmaceutical composition further includes a collagen, an extracellular matrix factor, a protein, or a polysaccharide.

The thrombolytic drug in the pharmaceutical composition can be selected from the group consisting of ticlopidine, warfarin, tissue plasminogen activator, eminase, retavase, streptase, tissue plasminogen activator, tenecteplase, abbokinase, kinlytic, urokinase, prourokinase, anisoylated purified streptokinase activator complex (APSAC), fibrin, and plasmin.

In some embodiments, the pharmaceutical composition further includes an angiogenic compound (e.g., vascular endothelial growth factor).

In another aspect, provided herein is a method of treating an ischemic tissue. The method includes administering the pharmaceutical composition described herein directly to the ischemic tissue in a subject, provided that the pharmaceutical composition is not administered intravenously.

In some embodiments, the ischemic tissue is an ulcer, or in a heart or limb in a subject. The ischemic tissue can be a muscle. In some embodiments, the subject has diabetes.

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a bar graph that shows the appearance scores of diabetic mice with lower limb ischemia treated with a pharmaceutical composition containing VEGF.

FIG. 2 is a bar graph that shows the blood flow of diabetic mice with lower limb ischemia treated with a pharmaceutical composition containing VEGF.

FIG. 3 is a bar graph that shows the appearance scores of diabetic mice with lower limb ischemia treated with a pharmaceutical composition containing ticlopidine.

FIG. 4 is a bar graph that shows the blood flow of diabetic mice with lower limb ischemia treated with a pharmaceutical composition containing ticlopidine.

FIG. 5 is a bar graph that shows the appearance scores of diabetic mice with lower limb ischemia treated with a pharmaceutical composition containing warfarin.

FIG. 6 is a bar graph that shows the blood flow of diabetic mice with lower limb ischemia treated with a pharmaceutical composition containing warfarin.

FIG. 7 is a graph showing the blood flow of diabetic mice with lower limb ischemia treated with a pharmaceutical composition containing warfarin.

FIG. 8 is a set of graphs showing functional analysis of diabetic mice with lower limb ischemia treated with a pharmaceutical composition containing warfarin.

FIG. 9 is a graph showing the appearance scores of diabetic mice with lower limb ischemia treated with a pharmaceutical composition containing warfarin at different time points after ischemia was created.

FIG. 10 is a graph showing the appearance scores of diabetic mice with lower limb ischemia treated with pharmaceutical compositions containing warfarin and hyaluronan of different molecular weights.

FIG. 11 is a graph showing the blood flow of diabetic mice with lower limb ischemia treated with pharmaceutical compositions containing warfarin and hyaluronan of different molecular weights.

FIG. 12 is a graph showing the appearance scores of diabetic mice with lower limb ischemia treated with pharmaceutical compositions containing warfarin and hyaluronan of similar viscosities.

### DETAILED DESCRIPTION

It was unexpectedly discovered that a pharmaceutical composition containing hyaluronan having certain viscosity and a thrombolytic drug was effective for treating ischemic tissues.

### Pharmaceutical Composition

Accordingly, described herein is a pharmaceutical composition for treating an ischemic tissue. The pharmaceutical composition includes a core component and a matrix component, the core component including a thrombolytic drug and the matrix component including a hyaluronan or derivative thereof. The pharmaceutical composition has a viscosity greater than 10 mPa·s. Depending on the parameters selected for measuring viscosity (e.g., the spindle and rotation speed), the viscosity of the composition may range from 10 to 10000 mPa·s (e.g., 10-100, 50-150, 100-200, 150-250, 250-500, 500-1000, 1000-1500, 1500-2000, 2000-2500, 2500-3000, 3000-3500, 3500-5000, 5000-6000, 6000-7000, 7000-8000, 8000-9000, or 9000-10000).

The viscosity of the pharmaceutical composition can fall within the range of the viscosities of 3 to 10 mg/ml (e.g., 3, 3.5, 4, 4.5, 5, 5.5, 6, 6.5, 7, 7.5, 8, 8.5, 9, 9.5, or 10 mg/ml) of hyaluronan having a mean molecular weight of 700 to 2000 kDa (e.g., 700, 800, 900, 1000, 1500, 1600, 1700, 1800, 1900, or 2000). Sec Tables 2-5 below. In some embodiments, the viscosity of the composition is the same as the viscosity of 5 mg/ml of hyaluronan having a mean molecular weight of 1560 kDa. For example, data described below show that 4 mg/ml of 2000 kDa hyaluronan, 5 mg/ml of 1,560 kDa hyaluronan, and 10 6.5 mg/ml 700 kDa hyaluronan have about the same viscosity.

The molecular weight of the hyaluronan in the pharmaceutical composition can range from 4 kDa to 5000 kDa (e.g., 4 to 20, 20 to 100, 100 to 500, 500 to 1000, 1000 to 2000, 2000 to 2500, 2500 to 5000, 5, 10, 50, 100, 200, 300, 400, 500, 750, 1000, 1500, 1800, 2000, 2500, 3000, 3500, 4000, 4500, or 5000 kDa). The concentration of the hyaluronan in the pharmaceutical composition can be 1 to 100 mg/ml (e.g., 1, 1.5, 2, 2.5, 3, 3.5, 4, 4.5, 5, 5.5, 6, 6.5, 7, 7.5, 8, 8.5, 9, 9.5, 10, 20, 30, 40, 50, 60, 70, 80, 90, or 100 mg/ml). In particular, the concentration of the hyaluronan in the pharmaceutical composition can be 3 to 10 mg/ml (e.g., 3, 3.5, 4, 4.5, 5, 5.5, 6, 6.5, 7, 7.5, 8, 8.5, 9, 9.5, or 10 mg/ml) if using hyaluronan having a mean molecular weight of 700 to 2000 kDa. A skilled practitioner would be able to select the appropriate combination of molecular weight and concentration to achieve a composition having the desired viscosity. A skilled practitioner would also be able to determine the viscosity of a composition using methods known in the art and commercially available instruments.

The term “hyaluronan” refers to a naturally-occurring anionic, non-sulfated glycosaminoglycan including repeated disaccharide units of N-acetylglucosamine and D-glucuronic acid, and its derivative. Naturally-occurring hyaluronan (also known as hyaluronic acid or hyaluronate) can be isolated from its natural sources, e.g., capsules of Streptococci, rooster comb, cartilage, synovial joints fluid, umbilical cord, skin tissue and vitreous of eyes, via conventional methods. See, e.g., Guillermo Lago et al. Carbohydrate Polymers 62(4): 321-326, 2005; and Ichika Amagai et al. Fisheries Science 75(3): 805-810, 2009. Alternatively, it can be purchased from a commercial vendor, e.g., Genzyme Corporation, Lifecore Biomedical, LLC and Hyaluron Contract Manufacturing. Derivatives of naturally-occurring hyaluronan include, but are not limited to, hyaluronan esters, adipic dihydrazide-modified hyaluronan, hyaluronan amide products, crosslinked hyaluronic acid, hemiesters of succinic acid or heavy metal salts thereof hyaluronic acid, partial or total esters of hyaluronic acid, sulphated hyaluronic acid, N-sulphated hyaluronic acid, and amines or diamines modified hyaluronic acid. They can be obtained by chemically modifying one or more of its functional groups (e.g., carboxylic acid group, hydroxyl group, reducing end group, N-acetyl group). A carboxyl group can be modified via esterification or reactions mediated by carbodiimide and bishydrazide. Modifications of hydroxyl groups include, but are not limited to, sulfation, esterification, isourea coupling, cyanogen bromide activation, and periodate oxidation. A reducing end group can be modified by reductive amination. It also can be linked to a phospholipid, a dye (e.g., a fluorophore or chromophore), or an agent suitable for preparation of affinity matrices. Derivatives of naturally-occurring hyaluronan can also be obtained by crosslinking, using a crosslinking agent (e.g., bisepoxide, divinylsulfone, biscarbodiimide, small homobifunctional linker, formaldehyde, cyclohexyl isocyanide, and lysine ethyl ester, metal cation, hydrazide, or a mixture thereof) or via internal esterification, photo-crosslinking, or surface plasma treatment. To make a hyaluronan solution, hyaluronan can be dissolved in a phosphate buffer solution (e.g., ≤0.05 M at pH 7±1) and/or NaCl (e.g., ≤0.9%).

The matrix component can contain one or more other matrix molecules, so long as the viscosity of the composition stays within the desired range. The matrix molecules can include gelatin, collagen, hyaluronan, fibronectin, elastin, tenacin, laminin, vitronectin, polypeptides, heparan sulfate, chondroitin, chondroitin sulfate, keratan, keratan sulfate, dermatan sulfate, carrageenan, heparin, chitin, chitosan, alginate, agarose, agar, cellulose, methyl cellulose, carboxyl methyl cellulose, glycogen and derivatives thereof. In addition, the matrix component can include fibrin, fibrinogen, thrombin, polyglutamic acid, a synthetic polymer (e.g., acrylate, polylactic acid, polyglycolic acid, or poly(lactic-co-glycolic acid), or a cross-linking agent (e.g., genipin, glutaraldehyde, formaldehyde, or epoxide).

The thrombolytic drug can be ticlopidine, warfarin, tissue plasminogen activator (t-PA), eminase (anistreplase), retavase (reteplase), streptase (streptokinase, kabikinase), activase, tenecteplase (TNKase), abbokinase, kinlytic (rokinase), urokinase, prourokinase, anisoylated plasminogen streptokinase activator complex (APSAC), fibrin, plasmin. The pharmaceutical composition can include one or more thrombolytic drugs. The pharmaceutical composition can contain the thrombolytic drugs at dosages similar to or lower than recommended clinical dosages.

The pharmaceutical composition can further include an angiogenic compound such as vascular endothelial growth factor (VEGF).

### Treatment Method

An effective amount of the pharmaceutical composition can be administered to a patient to treat an ischemic tissue. It can be administered (e.g., injected or applied) directly to or near the ischemic tissue (e.g., a muscle). The composition, which is gelatinous or viscous in consistency, is not administered intravenously.

The composition can be administered to a subject as needed, e.g., 1 to 5 times daily, 1 to 5 times per week, 1 to 5 times per month, for a suitable treatment period, e.g., 1 to 4 week, 1 to 12 months, or 1 to 3 years. It is preferable that it is administered as soon as possible after the ischemia or the ischemic damage has occurred (e.g., within 0 to 48 hours or 1-7 days).

The amount of the pharmaceutical composition administered should be sufficient to provide an effective dose of the therapeutic compound, e.g., a thrombolytic drug. An effective dose can be, for example 0.00001 to 10 μg (e.g., 0.00001 to 0.001, 0.001 to 0.005, 0.005 to 0.01, 0.05 to 0.1, 0.1 to 0.5, 0.5 to 1, 0.00001, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 μg) per gram of the body weight of the subject, depending on the efficacy of the thrombolytic drug.

“Treating” refers to administration of a pharmaceutical composition to a subject, who is suffering from or is at risk for developing a disorder, with the purpose to cure, alleviate, relieve, remedy, delay the onset of, prevent, or ameliorate the disorder, the symptom of the disorder, the disease state secondary to the disorder, or the predisposition toward the disorder. An “effective amount” refers to an amount of the composition that is capable of producing a medically desirable result in a treated subject. The treatment method can be performed alone or in conjunction with other drugs or therapies. The subject to be treated can be a human or a laboratory or domestic animal.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are herein incorporated by reference in their entirety.

### Example 1: Diabetic Lower Limb Ischemia Mouse Model

C57BL/6 male mice were sourced from the National Cheng Kung University (NCKU) Laboratory Animal Center or BioLASCO Taiwan Co., Ltd. They were housed in the animal facility of the NCKU Institute of Biotechnology for at least one week in order for them to adapt to the environment before experiments were performed. All the experiments performed were pre-approved by the Institutional Animal Care and Use Committees (IACUCs) at the NCKU.

This experimental animal model was established for studying therapeutics treatments for lower limb ischemia. Mice that were 6 months or older were treated with 50 mg/kg body weight of a streptozotocin (STZ) solution to induce type I diabetes in order to exhibit the characteristics of older age and slow-to-heal wounded diabetic tissues. Since low blood sugar levels in mice would interfere with the results, mice with blood sugar levels within the range of 400 mg/dl to 550 mg/dl were used for the experiments, and minute amount of insulin might be applied to mice to avoid life-threatening high blood sugar levels. In order to avoid the possibility of self-regenerative neovascularization, the femoral artery and its peripheral blood vessels in the lower limbs of the mice were severed. The model minimized the possibility of blood vessel regeneration, which allowed a more accurate assessment of the angiogenic ability of testing drugs.

To induce lower limb ischemia, a shaved diabetic mouse was placed in a gas anesthesia box with a ventilating gas that contained 1-3% isoflurane per liter of gas per minute. After the mouse was unconscious, it was moved to the surgical table and maintained under gas anesthesia. After fixing the limbs using breathable tapes, the mouse's body temperature was kept constant with a 37° C. heating pad. After the lower abdomen and limb of the mouse were disinfected, the skin of the limb was cut from a small opening at the left ankle to the thigh. Both ends of the two lateral vessels on the dorsal side of the mouse calf muscle were tied with surgical sutures, and the blood vessels were removed to block the blood flow of the dorsal vessels. The side branches and main vessels of the ventral femoral artery were then blocked. The end of the artery near the ankle and its surrounding vessels were tied by surgical suture to ensure that the femoral artery and peripheral blood flow were completely blocked.

After truncating the blood vessels, a pharmaceutical composition to be tested for its therapeutic effect on ischemia was applied on a tissue directly or injected into the gastrocnemius muscle at eight sites, and the surgical opening was sutured. The mouse was subcutaneously injected with 1 mg/kg body weight of ketorolac analgesic and lidocaine-HCl local anesthetic, and also administered with 1 ml of saline solution to relieve pain and provide hydration. Whenever necessary, a glucose solution was administered to maintain physical strength.

At day 0, 1, 2, 3, 4, 5, 6, 7, 14, 21 and 28 post-surgery, the apparent appearance and blood flow of the lower limb in the mice were evaluated using the score system shown in Table 1 and laser Doppler flowmetry, respectively. ROI was calculated as the ratio of the blood flow signal of the left lower limb to that of the untreated right lower limb post-surgery, and the ratio in percentage was normalized based on the blood flow signal taken before the operation.

TABLE 1
Lower limb ischemia appearance score of a diabetic mouse.

| Score | Condition/Appearance |
|---|---|
| 0 | Thigh amputation |
| 1 | Thigh necrosis |
| 2 | Calf amputation |
| 3 | Calf necrosis |
| 4 | Ankle joint necrosis |
| 5 | Claw amputation |
| 6 | Claw necrosis |
| 7 | Multiple toes amputation |
| 8 | One toe amputation |
| 9 | Multiple toes necrosis |
| 10 | One toe necrosis |
| 11 | Multiple blackened toes |
| 12 | One blackened toe |
| 13 | Multiple blackened/broken nails |
| 14 | One blackened/broken nail |
| 15 | Normal |

### Example 2: Hyaluronan Viscosity Test

Compositions containing different concentrations of hyaluronan with various molecular weights were produced.

The viscosity of the pharmaceutical compositions was tested using a DV2TRV Viscometer (Brookfield, USA) according to the manual. An appropriate spindle (CPE40 or CPE52) was selected according to the viscosity. Before testing, the machine was calibrated and set to run for 1 minute at 25° C. with 20 rpm. 500 μl of each sample was transferred with a viscosity pipette to the sample plate and the run button was pressed to start determining the viscosity of the sample. The viscosities of the hyaluronan at 5 mg/ml with mean molecular wrights of 1,560 kDa, 700 kDa and 2,000 kDa were determined and the results are shown in Table 2. The viscosity of 5 mg/ml of hyaluronan with mean molecular wrights of 1,560 kDa was used as the reference, and the viscosities of various concentrations of hyaluronan with mean molecular wrights of 700 kDa and 2,000 kDa were measured as shown in Tables 3 and 4. The concentrations of hyaluronan with mean molecular wrights of 700 kDa and 2,000 kDa at a viscosity close to that of the reference viscosity were then calculated and adjusted to 6.5 and 4 mg/ml respectively.

As shown in Table 5, it was noticeable that the viscosity of hyaluronan with the same concentration and molecular weight range was changed when the measuring parameter changed.

TABLE 2
Viscosity of 5 mg/ml of hyaluronan
with different molecular weights

| 5 mg/ml of | mean molecular wright of hyaluronan | | |
|---|---|---|---|
| hyaluronan (20 rpm) | 2000 kDa | 1560 kDa | 700 kDa |
| Viscosity (mPa · s) | 137.30 | 86.33 | 41.20 |
| Torque (%) | 84 | 52.8 | 25.2 |
| Shear stress (dyne/cm²) | 206.0 | 129.5 | 61.8 |

| | |
|---|---|
| Shear rate (1/s) | 150.0 |

TABLE 3
Viscosity of hyaluronan at different concentrations
with mean molecular weight of 700 kDa

| ~700 kDa of | Concentration of hyaluronan | | | |
|---|---|---|---|---|
| hyaluronan (20 rpm) | 5 mg/ml | 6 mg/ml | 7 mg/ml | 8 mg/ml |
| Viscosity (mPa · s) | 41.20 | 66.87 | 106.3 | 138.8 |
| Torque (%) | 25.2 | 40.9 | 65.0 | 84.9 |
| Sheer stress (dyne/cm²) | 61.8 | 100.3 | 159.4 | 208.2 |

| | |
|---|---|
| Shear rate (1/s) | 150.0 |

TABLE 4
Viscosity of hyaluronan at different concentrations
with mean molecular weight of 2000 kDa

| ~2000 kDa of | Concentration of hyaluronan | | | | |
|---|---|---|---|---|---|
| hyaluronan (20 rpm) | 1 mg/ml | 2 mg/ml | 3 mg/ml | 4 mg/ml | 5 mg/ml |
| Viscosity (mPa · s) | 7.36 | 20.27 | 52.16 | 87.15 | 137.3 |
| Torque (%) | 4.5 | 12.4 | 31.9 | 53.3 | 84 |
| Sheer stress (dyne/cm²) | 11.04 | 30.41 | 78.23 | 130.7 | 206.0 |

| | |
|---|---|
| Shear rate (1/s) | 150.0 |

TABLE 5
Viscosity of hyaluronan at different concentrations
with a mean molecular weight of 1~1.8 MDa

| Viscosity of | | | | | | | | |
|---|---|---|---|---|---|---|---|---|
| hyaluronan | | Concentration of hyaluronan (mg/ml) | | | | | | |
| (mPa · s) | | 1 | 2 | 3 | 4 | 5 | 6 | 7 |
| 50 | rpm | 16.6 | 42.0 | 61.7 | 89.6 | 121.1 | — | — |
| | | Y = 25.66X-10.77, R² = 0.9935 | | | | | | |
| 10 | rpm | 47.1 | 82.4 | 151.7 | 302.8 | 427.7 | 564.4 | — |
| | | Y = 130.8X-222.37, R² = 0.9993 | | | | | | |
| 2 | rpm | 127.5 | 189.7 | 349.9 | 977.7 | 1436 | 1995 | 2596 |

Y = 541.39X-1226.5, R² = 0.9964

### Example 3: Pharmaceutical Composition Containing Vascular Endothelial Growth Factor (VEGF)

A composition (DIV) containing 5 mg/ml of hyaluronan with a mean molecular weight of 1,560 kDa and VEGF was administered to the mice and their effects on the lower limbs and blood flow were evaluated as described in Example 1 above. Diabetic mice not treated with the composition after the surgery were used as controls. VEGF drugs have been described to have an angiogenic effect in the literature. The maximum and minimum effective doses of VEGF in humans were converted to doses for mice according to body weight.

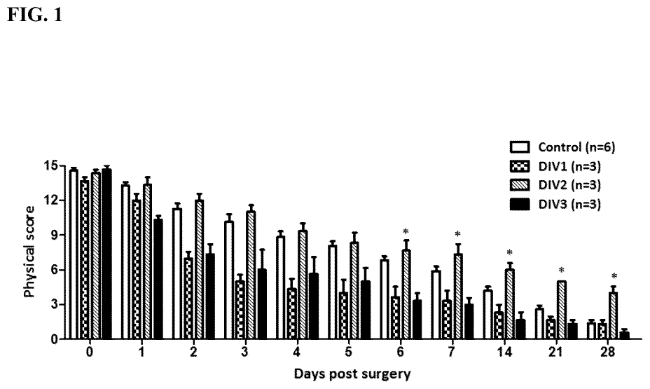

The appearance scores are shown in FIG. 1. Administering 100 μl of DIV amounted to giving the mice 3.125 ng/g body weight of VEGF. Administering 3.125 ng/g of VEGF (DIV2) to the mice resulted in higher appearance scores compared with the control group. When the dose of VEGF was lowered to 0.3 ng/g (DIV1), it was observed that the appearance scores were lower than those of the control group. When the dose of VEGF was increased to 15 ng/g (DIV3), it was observed that the ischemia and gangrene of the lower limbs worsened.

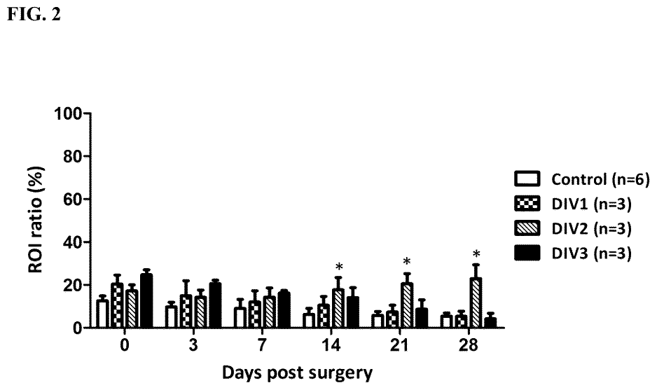

Results of the blood flow measurements are shown in FIG. 2. As compared with the control group, only 3.125 ng/g of VEGF significantly increased the blood flow at day 14 and then on post surgery. The results showed VEGF was only effective at specific dosages between 0.3 ng/g and 15 ng/g.

### Example 4: Pharmaceutical Composition Containing Ticlopidine

A composition (DIT) containing 5 mg/ml of hyaluronan with a molecular weight range 1000 to 1800 kDa and ticlopidine was administered to the mice and their effects on the lower limbs and blood flow were evaluated as described in Example 1 above. Diabetic mice not treated with the composition after the surgery were used as controls. The maximum and minimum effective doses of ticlopidine in humans were converted to doses for mice according to body weight.

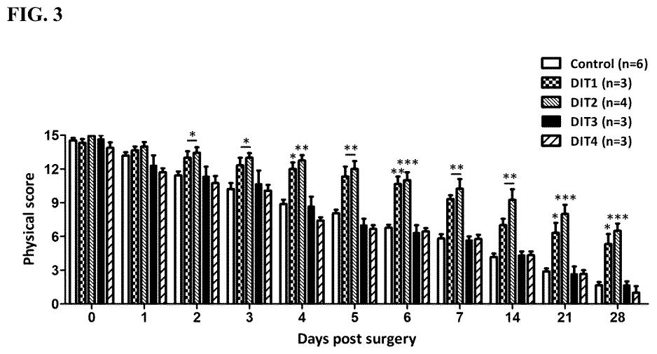

The post-operative appearance scores are shown in FIG. 3. Administering 100 μl of DIT amounted to giving the mice 0.7 μg/g body weight of ticlopidine (DIT2). At that dose, the appearance scores from day 2 to day 28 were significantly different from those in the control group (P<0.05). The appearance scores were also significantly different from those in the control group when 0.07 μg/g of ticlopidine (DIT1) was administered.

However, when the dose was increased to 7 μg/g body weight (DIT3) or 110 μg/g body weight (DIT4), the appearance scores were not significantly different from those of the control group during the observation period. The results also showed that ticlopidine was able to effectively alleviate the gangrene caused by ischemia in lower doses, but the effect was reduced when the dose was below a certain threshold.

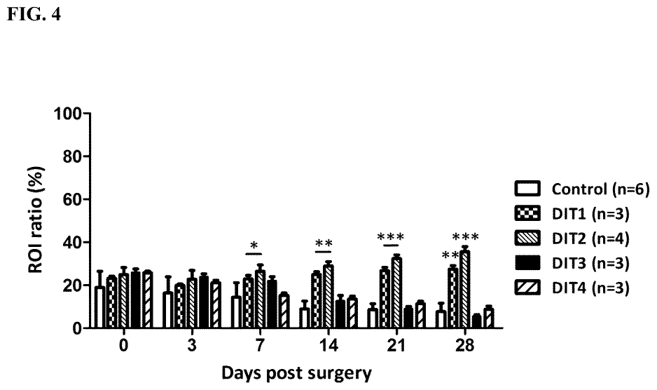

Results of the blood flow measurements are shown in FIG. 4. The results showed that at 0.07 μg/g (DIT1) and 0.7 μg/g (DIT2) body weight of ticlopidine, the blood flow signals in the ischemic lower limb increased significantly as compared to the control from day 7 post surgery (P<0.05). However, at the higher doses (DIT3 and DIT4), there was no significant difference in blood flow as compared to the control.

### Example 5: Pharmaceutical Composition Containing Warfarin

A composition (DIW) containing 5 mg/ml of hyaluronan with a mean molecular weight of 1,560 kDa and warfarin was administered to the mice and their effects on the lower limbs and blood flow were evaluated as described in Example 1 above. Diabetic mice not treated with the composition after the surgery were used as controls. The maximum and minimum effective doses of warfarin in humans were converted to doses for mice according to body weight.

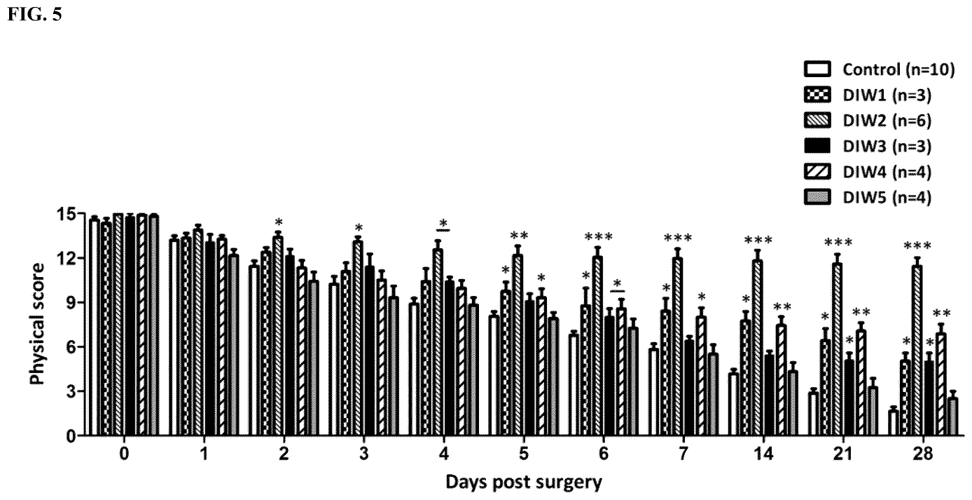

The post-operative appearance scores are shown in FIG. 5. Administering 100 μl of DIW amounted to giving the mice 70 ng/g body weight of warfarin. At this dosage (DIW2), the appearance scores from day 2 to day 28 were most significantly different from those in the control group (P<0.05). In addition, when the dose was increased two times to 140 ng/g body weight (DIW4), the appearance scores from day 5 to day 28 were also significantly different from those in the control group (P<0.001-0.05). On the other hand, if the dose increased by a factor of three to 210 ng/g body weight (DIW5), the appearance scores were not significantly different from those of the control group. The results suggested that the optimal dose of DIW is 70 ng/g body weight of warfarin, which maintained the appearance integrity of the lower limb and avoided tissue gangrene in the case of ischemic distress.

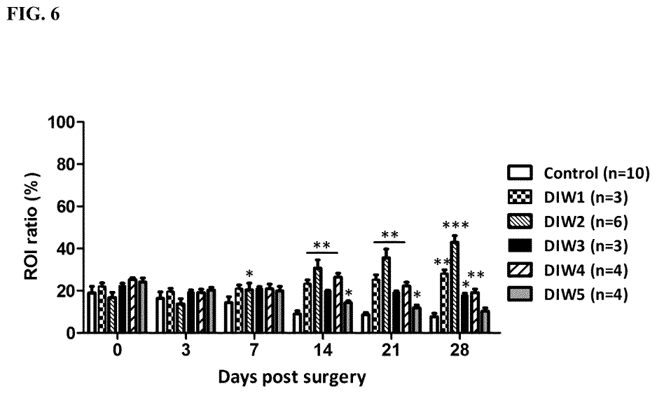

Results of the blood flow measurements are shown in FIG. 6. The results showed that when the dose of warfarin was 70 ng/g body weight (DIW2), the blood flow signals of the lower limbs were significantly different from those of the control group starting from day 7 after surgery. When the dose was 35 ng/g (DIW1), 105 ng/g (DIW3), 140 ng/g (DIW4), or 210 ng/g (DIW5), the lower limb blood flow signals were significantly different from those of the control group starting from day 14 post surgery. However, at 210 ng/g of warfarin, there was no significant difference at day 28 post surgery as compared with the control group.

Comparing the results obtained with DIV (Example 3), DIT (Example 4), and DIW, DIW administer at 70 ng/g body weight of warfarin (DIW2) appeared to be the most effective. It was observed that in the DIW2 group, only the distal ends of the toes of the lower limbs were slightly blackened during the postoperative observation period. By contrast, in the control group, the left lower limb has a blackening appearance at day 3 after surgery. Also, some tissue shedding could be observed on day 7 post surgery, and gangrenes in the lower limb could be observed at day 14 post surgery. Thus, the appearance of gangrene caused by ischemia could be significantly alleviated by administration of DIW2.

In addition, the distribution of the blood flow signals detected by laser Doppler showed that the blood flow signals in the DIW2 group gradually increased after day 14 post surgery. Conversely, no increase in blood flow signals was observed in the control group. In addition, due to gangrenes in the lower limbs, the laser Doppler imager was unable to detect the blood flow of the lower limbs in the control group. The results further showed that appearance of gangrene in the lower limbs was significantly reduced in the DIW2 group as compared to the control group.

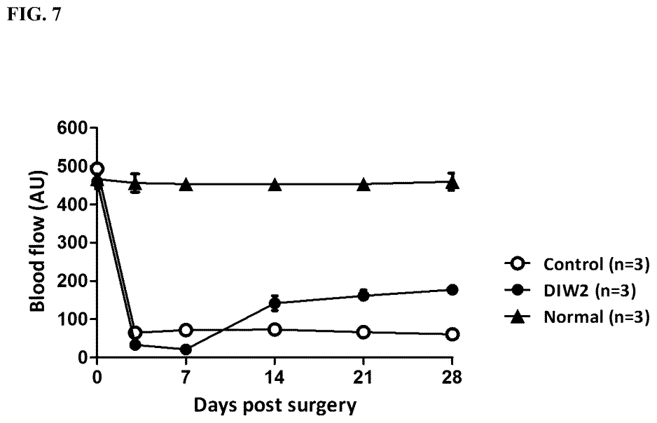

Further analysis of blood flow changes in the lower limbs after surgery in the control and DIW2 groups was carried out using an oximeter. As shown in FIG. 7, blood flow in the lower limbs in the control group and the DIW2 group began to decline right after surgery. The DIW2 group began to recover blood flow on day 7 post surgery, and the blood flow reached about 100-200 AU during the observation period. The control group did not show any blood flow recovery during the 28-day observation period, and actually showed a slight decrease.

### Example 6: Functional Evaluation of Mice Treated with a Pharmaceutical Composition Containing Warfarin

The mice in the DIW2 group and control group were further evaluated functionally. The evaluation was performed on day 35 post surgery.

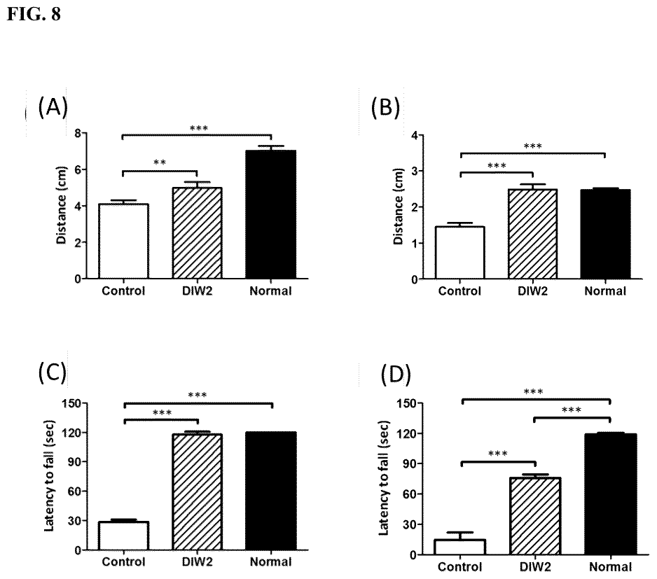

Each mouse was placed on a platform and the tail was pulled at a fixed height of about 5 cm to observe its standing grip pose. It was observed that, in the standing posture, the mice in the DIW2 group still could not grasp as well as normal mice. Nevertheless, it was found that the stride length and sway length of the DIW group were significantly increased compared with those of the control group (P<0.001) and comparable to those of the normal mice. See FIGS. 8(A) and (B). The results showed that, as the lower limbs of the mice in the DIW2 group started to atrophy after the surgery, their steps could not completely return to normal, but they were still significantly better compared to the control group.

In addition, the mice were placed on a running track to analyze their gait according to their footprints. It was observed that, at 5 rpm, the mice in the DIW2 group and the normal mice were able to stay on the track without falling for a similar period of time, while the mice in the control group fell off after a significantly shorter period. See FIG. 8(C). When the rotational speed was increased to 10 rpm, it was observed that the mice in the DIW2 group fell off much earlier than the normal mice, but still stayed on significantly longer than the control mice. See FIG. 8(D).

### Example 7: Timing Effect of Treatment

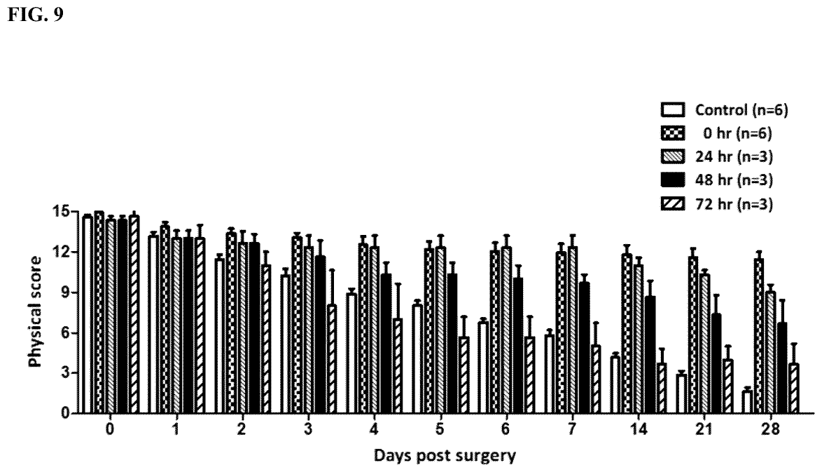

Diabetic mice with lower limb ischemia were produced as described in Example 1. The mice were treated with a composition containing warfarin and 5 mg/ml of hyaluronan with molecular weight range 1000 to 1800 kDa at a dose of 70 ng/g body weight of warfarin like the mice in the DIW2 group described above, but at different time points after surgery. As shown in FIG. 9, it was observed that, if the treatment was delayed after surgery, the lower limb appearance scores decreased in a manner that depended on the length of the delay. Nevertheless, even if the treatment was administered as late as 48 hours after surgery, the appearance score was still about 9 points on day 28 post surgery, indicating that part of the limb still remained with necrosis of the toes only. If the treatment was delayed for 72 hours, necrosis of the lower limb could not be rescued.

### Example 8: Effect of Different Molecular Weights of Hyaluronan on Treatment Efficacy

Whether different molecular weights of hyaluronan in the pharmaceutical composition affected therapeutic effect was investigated.

Compositions containing warfarin and 5 mg/ml of hyaluronan at different mean molecular weights, i.e., 74 kDa, 357 kDa, 700 kDa, 1560 kDa, 2000 kDa, and 2590 kDa were produced. The compositions were administered to diabetic mice with lower limb ischemia at a dose of 70 ng/g body weight of warfarin and evaluated as described in Example 1.

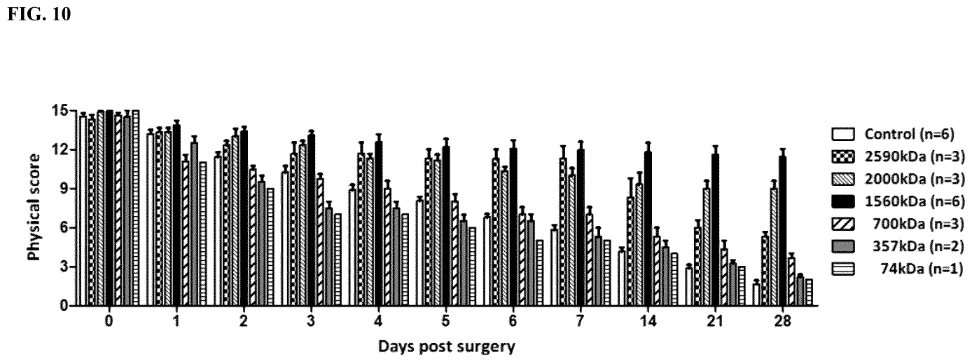

As shown in FIG. 10, the composition containing hyaluronan with mean molecular weight of 1,560 kDa exhibited the best therapeutic effect. As the mean molecular weight increased to 2,000 kDa, the appearance scores were better than those of the control group, but lower than those of 1,560 kDa hyaluronan. In addition, hyaluronan having mean molecular weights below 1,560 kDa tended to decrease the appearance scores. The appearance scores in the 357 kDa and 74 kDa groups at 28 days after surgery were deteriorating as compared with the control group.

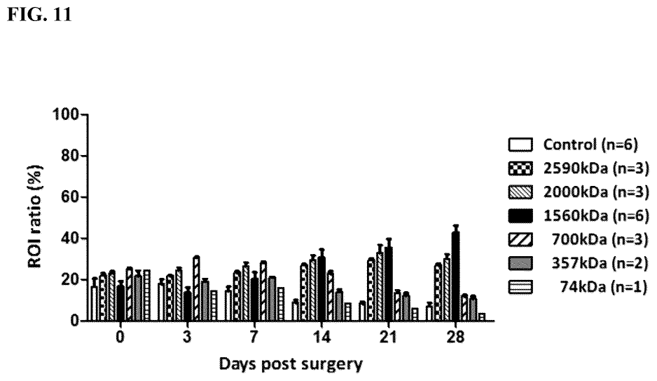

As shown in FIG. 11, the blood flow signals of the 2,000 kDa and 1,560 groups were significantly increased after day 14 post surgery as compared with the control group.

### Example 9: Effect of Viscosity on Treatment Efficacy

Whether viscosity affected therapeutic effect of the pharmaceutical compositions was investigated.

Compositions each containing 4 mg/ml of mean 2000 kDa of hyaluronan, 5 mg/ml of mean 1,560 kDa of hyaluronan, or 6.5 mg/ml of mean 700 kDa of hyaluronan were produced. The concentrations of hyaluronan were selected such that all three had a similar viscosity. See Tables 2, 3, and 4 above. Each was mixed with warfarin to produce a gelatinous composition. The compositions were administered to diabetic mice with lower limb ischemia at a dose of 70 ng/g body weight of warfarin and evaluated as described in Example 1.

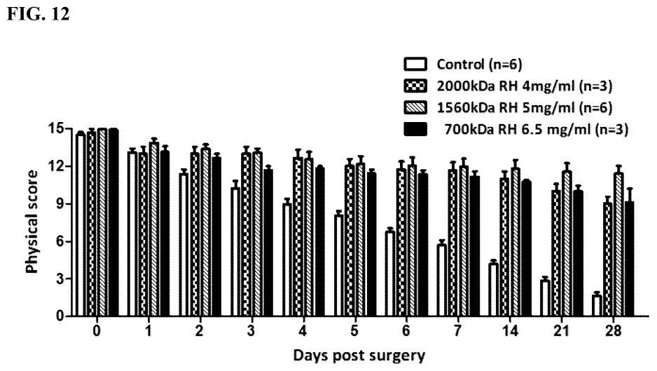

As shown in FIG. 12, the lower limb appearance scores among the mice treated with compositions containing hyaluronan of different molecular weights were not very different during the observation period. Thus, the viscosity of the hyaluronan appeared to be more critical than its molecular weight. Note, as shown in FIGS. 10 and 11, 5 mg/ml of mean 1560 kDa hyaluronan and 5 mg/ml of mean 2000 kDa hyaluronan were significantly more effective than hyaluronan of higher or lower molecular weights at the same concentration. These results also suggest that a certain range of viscosity is optimal.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A pharmaceutical composition for treating an ischemic tissue, comprising: a core component and a matrix component, wherein the core component includes a thrombolytic drug and the matrix component includes a hyaluronan or derivative thereof, the pharmaceutical composition having a viscosity greater than 10 mPa·s and up to 10000 mPa·s.

2. The pharmaceutical composition of claim 1, wherein the hyaluronan has a mean molecular weight of 100 kDa to 5000 kDa.

3. The pharmaceutical composition of claim 2, wherein the hyaluronan has a mean molecular weight of 700 kDa to 2000 kDa.

4. The pharmaceutical composition of claim 1, wherein the pharmaceutical composition contains 1 mg/ml to 100 mg/ml of the hyaluronan.

5. The pharmaceutical composition of claim 1, wherein the viscosity is within the range of viscosities of 3 to 10 mg/ml of hyaluronan having a mean molecular weight of 700 to 2000 kDa.

6. The pharmaceutical composition of claim 1, wherein the viscosity is the same as the viscosity of 5 mg/ml of hyaluronan having a mean molecular weight of 1560 kDa.

7. The pharmaceutical composition of claim 5, wherein the mean molecular weight of the hyaluronan is 700 to 2000 kDa and the concentration of the hyaluronan is 3 to 10 mg/ml.

8. The pharmaceutical composition of claim 6, wherein the mean molecular weight of the hyaluronan is 1560 kDa and the concentration of the hyaluronan is 5 mg/ml.

9. The pharmaceutical composition of claim 1, wherein the matrix component further includes a collagen, an extracellular matrix factor, a protein, or a polysaccharide.

10. The pharmaceutical composition of claim 1, wherein the thrombolytic drug is selected from the group consisting of ticlopidine, warfarin, tissue plasminogen activator, eminase, retavase, streptase, tissue plasminogen activator, tenecteplase, abbokinase, kinlytic, urokinase, prourokinase, anisoylated purified streptokinase activator complex (APSAC), fibrin, and plasmin.

11. The pharmaceutical composition of claim 1, further comprising an angiogenic compound.

12. The pharmaceutical composition of claim 11, wherein the angiogenic compound is vascular endothelial growth factor (VEGF).

13. The pharmaceutical composition of claim 1, wherein the pharmaceutical composition is directly administered to the ischemic tissue but is not administered intravenously.

14. The pharmaceutical composition of claim 13, wherein the ischemic tissue is an ulcer, or in a heart or limb in a subject.

15. The pharmaceutical composition of claim 14, wherein the ischemic tissue is a muscle.

16. A method of treating an ischemic tissue in a subject, comprising:
administering a pharmaceutical composition directly to the ischemic tissue, provided that the pharmaceutical composition is not administered intravenously;
wherein the pharmaceutical composition contains a core component and a matrix component, the core component including a thrombolytic drug and the matrix component including a hyaluronan or derivative thereof, and wherein the pharmaceutical composition has a viscosity greater than 10 mPa·s and up to 10000 mPa·s.

17. The method of claim 16, wherein the ischemic tissue is an ulcer, or in a heart or limb in a subject.

18. The method of claim 17, wherein the ischemic tissue is a muscle.

19. The method of claim 16, wherein the subject has diabetes.

20. The pharmaceutical composition of claim 16, wherein the hyaluronan has a mean molecular weight of 100 kDa to 5000 kDa.
